(19) United States
(12) Patent Application Publication
(10) Pub. No.: US 2025/0387110 A1
(43) Pub. Date: Dec. 25, 2025

(54) IMPLANTABLE CLOSURE DEVICE

(71) Applicant: W. L. Gore & Associates, Inc., Newark, DE (US)

(72) Inventors: Gilbert R. Gabo, Flagstaff, AZ (US); Jeffrey B. Duncan, Flagstaff, AZ (US)

(21) Appl. No.: 19/243,118

(22) Filed: Jun. 19, 2025

Related U.S. Application Data
(60) Provisional application No. 63/661,792, filed on Jun. 19, 2024.
(60) Provisional application No. 63/661,787, filed on Jun. 19, 2024.
(60) Provisional application No. 63/825,863, filed on Jun. 18, 2025.

(51) Int. Cl. A61B 17/00

(52) U.S. Cl. CPC A61B 17/0057; A61B 2017/00004; A61B 2017/00623; A61B 2017/00628; A61B 2017/0065

(57) ABSTRACT

An implantable closure device includes a body comprising a bioabsorbable material and defining generally a disk-shape having a hole. The body is distensible so as to define the hole having an open position when the hole is presented with a force and a closed position when the force is absent.

### CROSS REFERENCE TO RELATED APPLICATION

The present application claims the benefit of U.S. Provisional Application No. 63/661,792, filed Jun. 19, 2024, U.S. Provisional Application No. 63/661,787, filed Jun. 19, 2024, and U.S. Provisional Application No. 63/825,863, filed Jun. 18, 2025, which are incorporated herein by reference in their entireties for all purposes.

### FIELD

The present disclosure relates generally to apparatuses, systems, and methods relating to closure devices. Specifically, the disclosure relates to apparatuses, systems, and methods relating to closure devices to be implanted in a body, such as in a blood vessel.

### BACKGROUND

Vascular closure devices are vital components of a vascular surgery, such as for endovascular aneurysm repair, transcatheter aortic valve implantation, and/or providing percutaneous circulatory support. After the surgery which involves penetrating the skin and the underneath tissue to access a blood vessel or other vasculature, the access path needs to be closed. Commercially available vascular closure devices include suture-based (e.g., Prostar® and ProGlide® from Abbott Cardiovascular), collagen-based (e.g., MANTA® from Teleflex Inc.), patch-based (e.g., PerQseal® from Vivasure Medical), or membrane-based (e.g., InClosure device from InSeal Medical). There is a need for closure devices that can be easily implanted and are capable of providing an effective seal in the vasculature to prevent possible leakage or infection.

### SUMMARY

Closure devices are provided to have a body with a distensible hole through which an introducer may be received to allow the closure devices to be implanted. The closure devices as discussed herein may be used in various applications, including but not limited to surgical applications. For example, the closure devices may be used in conjunction with pusher sleeves/sheaths, introducers, guidewires, and/or any other suitable devices for introducing or implanting medical devices and apparatuses into and out of the body during surgical procedures.

According to one example (“Example 1”) an implantable closure device includes a body comprising a bioabsorbable material and defining generally a disk-shape having a hole. The body is distensible so as to define the hole having an open position when the hole is presented with a force and a closed position when the force is absent.

According to another example (“Example 2”) further to Example 1, the implantable closure device includes an adhesive applied to the body.

According to another example (“Example 3”) further to Example 2, the adhesive is a biocompatible adhesive.

According to another example (“Example 4”) further to any one of the preceding Examples, the implantable closure device further includes an extension weaved through the body and extending from the body, and actuating the extension causes the hole to be actuated between the open position (first configuration) and the closed position (second configuration).

According to another example (“Example 5”) further to Example 4, the extension comprises a network of interconnected fibrils.

According to another example (“Example 6”) further to Example 4, the extension comprises: a first portion with micro-pleats that is weaved through the body, and a second portion with macro-pleats that is extending from the body.

According to another example (“Example 7”) further to any one of the preceding Examples, the body is radially distensible.

According to another example (“Example 8”) further to Example 1, the bioabsorbable material has a property of stored length operable to effectuate recovery of the hole from the open position to the closed position.

According to another example (“Example 9”) further to Example 8, the stored length is facilitated by defining the bioabsorbable material with micro-pleats that allow for elongation and contraction.

According to one Example (“Example 10”), a surgical apparatus for implanting and closing a vasculature in a body includes: the implantable closure device of any one of the preceding Examples; an introducer operable to penetrate through a tissue region adjacent to the vasculature to form a tissue tract; and a pusher sleeve having a main channel operable to accept the introducer therein, the pusher sleeve having a first end and a second end. The second end is operable to engage the implantable closure device during implanting the implantable closure device adjacent to the vasculature to be closed.

According to another example (“Example 11”) further to Example 10, the pusher sleeve includes an openable slit extending longitudinally from the first end to the second end of the pusher sleeve operable to allow the passing of the introducer therethrough to facilitate the coupling of the pusher sleeve onto the introducer.

According to another example (“Example 12”) further to Example 10 or 11, the pusher sleeve includes an inner conduit that defines an inner channel extending longitudinally from the first end to the second end independent of and not in fluid communication with the main channel.

According to another example (“Example 13”) further to Example 12, the inner channel is operable to facilitate the transfer of an adhesive material to the implantable closure device.

According to another example (“Example 14”) further to any one of Examples 10-13 further to Example 6, the second portion of the extension extends through the tissue tract to facilitate cell ingrowth.

According to another example (“Example 15”) further to any one of Examples 10-14, the surgical apparatus further includes a guidewire operable to extend through the introducer.

According to one Example (“Example 16”), a method of implanting and closing a vasculature in a body includes: extending an introducer through a hole in an implantable closure device having a body comprising a bioabsorbable material, the hole operable to be distensible in one or more directions by the urging engagement of the introducer therein having a larger diameter into the hole having a smaller diameter; extending the introducer through a tissue tract formed in a tissue region adjacent to the vasculature; advancing the implantable closure device toward the vasculature via a pusher sleeve to adjacent to an in contact with the vasculature, the pusher sleeve having a main channel operable to be received onto the introducer therein; retracting the pusher sleeve from the tissue tract; and retracting the introducer from the tissue tract and implantable closure device causing the hole of the implantable closure device to self-transition from an open position when the introducer is located within the hole to a closed position when the introducer is withdrawn from the hole in an absence of a force being applied to open the hole.

According to another example (“Example 17”) further to Example 16, the retraction of the introducer from the tissue tract causes a second portion of an extension to extend from the implantable closure device through the tissue tract to facilitate cell ingrowth, and first portion of the extension is weaved through the body and across the hole such that the first portion of the extension extends in a cross-cross pattern to and from opposing sides of the hole of the implantable closure device.

According to another example (“Example 18”) further to Example 17, the method further includes tensioning the extension. The tensioning the extension is operable to cause the first portion of the extension to substantially close the hole of the implantable closure device.

According to another example (“Example 19”) further to any one of Examples 16-18, the pusher sleeve includes an openable slit extending longitudinally along the pusher sleeve. The method further includes opening the openable slit of the pusher sleeve to retrieve the pusher sleeve from the tissue tract independently of the introducer.

According to another example (“Example 20”) further to Example 16, the method further includes, prior to the retraction of the pusher sleeve, delivering an adhesive to the implantable closure device to couple the implantable closure device to the vasculature and/or adjacent tissue.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate some embodiments, and together with the description serve to explain the principles of the disclosure.

FIG. 1A shows a top view of a closure device in a substantially open configuration according to some embodiments disclosed herein;

FIG. 1B shows a top view of a closure device in a substantially closed configuration according to some embodiments disclosed herein;

FIG. 2A shows an angled view of a pusher sleeve according to some embodiments disclosed herein;

FIG. 2B shows a front view of a pusher sleeve according to some embodiments disclosed herein;

FIGS. 3A through 3F show cross-sectional view of a portion of the tissue region surrounding a vasculature during a procedure performed using the closure device and the pusher sleeve according to some embodiments disclosed herein;

FIG. 4A shows a top view of a closure device in a substantially open configuration according to some embodiments disclosed herein;

FIG. 4B shows a top view of a closure device in a substantially closed configuration according to some embodiments disclosed herein;

FIG. 5 shows a cross-sectional view of a portion of the tissue region surrounding a vasculature during a procedure performed using the closure device and the pusher sleeve according to some embodiments disclosed herein;

FIG. 6 shows a side view of a closure device in a substantially closed configuration according to some embodiments disclosed herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein “non-woven” generally refers to a type of fabric or substrate made directly from fibers or filaments or from a web of fibers without the preliminary filament preparation needed for weaving, knitting, or braiding.

As used herein “pleat” generally refers to a portion of fabric or substrate that is folded or otherwise positioned back on itself. The term as used herein does not require linear, uniform, overlapping, or even arrangements, although those are within the scope of the use of the term. Furthermore, a pleat need not be held by stitching, pressing or sewing, but may instead be held or defined by the material properties of the substrate.

As used herein “fold” generally refers to a portion of fabric or substrate at which the fabric or substrate has a change in shape to define the position and/or shape of a pleat.

As used herein “inelastic” generally refers to a material property in which the material substantially resists elongation or lengthening.

As used herein “micro-pleat” generally refers to a portion of material that is adjacent to at least on fold, where the portion of material has a width defined from or between the fold(s) of less than 1 mm.

As used herein “macro-pleat” generally refers to a portion of material that is adjacent to at least on fold, where the portion of material has a width defined from or between the fold(s) of 1 mm or greater.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

In various examples, closure devices according to this patent specification may be used in association with various medical procedures, including in association with endoluminal (e.g., endovascular procedures) that require access to one or more body lumens through an access site. The puncture, or wound, to the skin, tissue, and lumen proximate the access site may be treated through use of such closure devices. For example, the closure device may assist in short term wound closure and/or longer term healing.

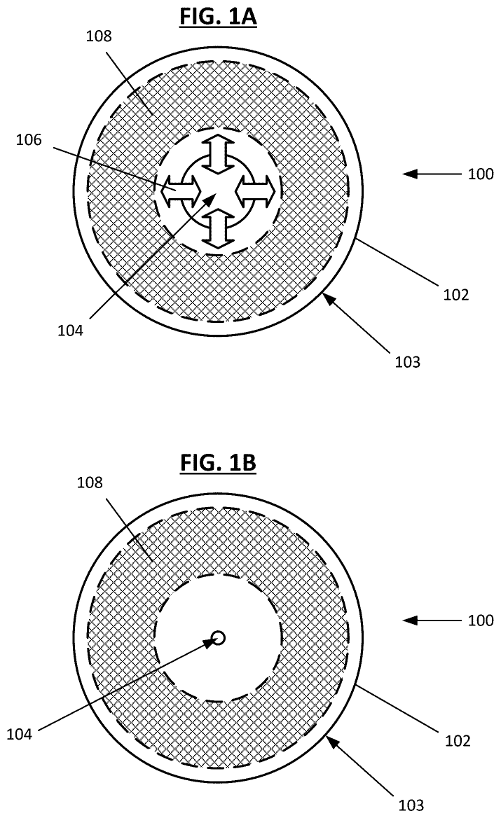

FIGS. 1A and 1B show an example of an implantable closure device 100 according to some embodiments disclosed herein, provided as an example of the various features of the device and, although the combination of those illustrated features is clearly within the scope of invention, that example and its illustration is not meant to suggest the inventive concepts provided herein are limited from fewer features, additional features, or alternative features to one or more of those features of the material shown in the figures and may include the other features such as being formed into three-dimensional structures such as shown in subsequent figures as disclosed herein. It should also be understood that the reverse is true as well. One or more of the components depicted in FIGS. 1A and 1B can be employed in addition to, or as an alternative to components depicted in other figures as discussed herein.

Discussed herein are devices and methods for closing an access site and access path into vasculature of a patient, such as an artery or a vein, which is formed through the skin and the tissue region adjacent to the vasculature. The skin and the tissue region are penetrated or otherwise opened to provide an access site corresponding to an access path to the vasculature. As shown in FIGS. 1A and 1B, the closure device 100 includes a body 102 defining a disk-shape having a hole 104 therethrough. The hole 104 may be located at any suitable section of the body 102, such as at the center or close to the center of the body 102. In various examples, the hole 104 is distensible (e.g., expandable) in one or more directions as shown by the arrows 106, in response to a force being applied to the hole 104. In some examples, the hole 104 may be radially distensible. In some examples, the body 102 has a portion including an adhesive 108. The adhesive 108 may be applied onto a surface of the body 102, at least partially imbibed into the body 102, or otherwise associated with the body 102 as desired. The adhesive 108 may be located on the body 102 between an outer periphery 103 of the body 102 and the hole 104. In some examples, the adhesive 108 may be applied at one or more locations surrounding the hole 104. In some examples, the adhesive 108 may be dry (or solid); in some examples, the adhesive 108 may be wet (or liquid).

FIG. 1A shows the device 100 in a first configuration (open position) in which the hole 104 is sufficiently opened to allow an introducer to pass through the hole 104 to allow the device 100 to be implanted at a desired location at or within the vasculature, as further explained herein. The first configuration may be referred to as a substantially open configuration, with the hole 104 being enlarged by the urging engagement of the introducer passing therethrough, such as by the urging engagement of the introducer having a larger diameter into the hole having a smaller diameter. FIG. 1B shows the device in a second configuration (closed position) in which the hole 104 is sufficiently closed to decrease the amount of fluid, tissue cells, or other body matter from passing through the hole 104 (relative to the first configuration), after the device 100 is implanted at the desired location and the introducer being removed from the hole 104. The second configuration may be referred to as a substantially closed configuration. As such, by definition, an unobstructed area of the hole 104 is greater in the first configuration than in the second configuration. In some examples, the device 100 automatically transitions from the first configuration to the second configuration in response to an absence of force being applied to the hole 104 such as by the introducer in urging engagement with the hole 104. As such, the device 100 may be referred to as a self-sealing or self-closing closure device when the body 102 is capable of self-transitioning from the first configuration to the second configuration, in the absence of a force applied to the hole 104.

It is to be understood that although FIGS. 1A and 1B illustrate the device 100 in a substantially disk-shaped configuration, it is to be understood that any suitable shape or configuration may be implemented for the device 100. For example, the shape of the device 100 may be polygonal, ovular, or lacking a clear outline, such as one having a “fuzzy”, fluffy, or indistinct outline that may be a result of the device 100 being formed by tearing off pieces from a material instead of cutting the pieces off using scissors. As such, it is to be understood that the device 100 may have an indistinct periphery instead of a well-defined periphery that can be observed by the naked eye.

It is also to be understood that the shape or configuration of the hole 104 may be different in the two configurations. For example, in FIG. 1B, the hole 104 may not be circular (e.g., ovular or polygonal). In FIG. 1A, the hole 104 may have a different shape, such as non-circular or other shape suited to accommodate the cross-sectional shape of the object being passed through the hole such as an introducer that is received through the hole 104, as further explained herein.

In some examples, the body 102 of the device 100 may be made of any suitable bioabsorbable material that facilitates or promotes tissue ingrowth after the device 100 is implanted. Bioabsorbable, as used herein, is a term that is used interchangeably with biodegradable, bio-resorbable, and resorbable, as well as other terms of art to describe the property of disintegration after in vivo implantation, as is understood in the art. For example, the bioabsorbable material may include but is not limited to: copolymers and homopolymers of poly (α-hydroxy esters), such as copolymers of poly(lactic-co-glycolic acid) (PLGA), poly(glycolic acid) (PGA), and poly(lactic acid) (PLA); trimethylene carbonate (TMC); copolymers of PLA and TMC (PLA:TMC), copolymers of PGA and TMC (PGA:TMC) and copolymers of PLGA and TMC; and combinations thereof. In some examples, the material of the body 102 may be biocompatible, antibacterial, anti-inflammatory, and/or conductive to the body's healing process. Various other materials may be implemented that exhibit certain properties for facilitating at least some of the properties and functionalities of the bioabsorbable material. At least some of the properties that facilitate the final functionality and properties of the bioabsorbable material are described herein in more detail.

The bioabsorbable material for the body 102 includes various properties suitable for the application in which it is applied. For example, the body 102 may be a woven or non-woven bioabsorbable member which includes from about 30% to about 130% stored length. In some examples, the bioabsorbable material may have the property of stored length operable to effectuate recovery of the hole 104 from the open position to the closed position. In some examples, the stored length is facilitated by defining the bioabsorbable material with micro-pleats that allow for elongation and contraction. The body 102 may be formed as a clump or network of fibrils or filaments. The amount of stored length is tunable during processing to facilitate the ability to provide for different amount of stretch or expansion during use. In some examples, the surface texture of the body 102 may be defined by pleats (e.g., macro-pleats and micro-pleats). In some examples, the body 102 may include a melt blown fabric material. In some examples, negative thermal expansion (NTE) or negative expansion (such as by about 50%) of the fabric material of the body 102 causes the pleats to form on the surfaces of the body 102. In various examples, the surface texture of the body 102 may include macro-pleats, micro-pleats, or both macro-pleats and micro-pleats.

The pleating facilitates storing length of the body 102 by positioning the body 102 in such a way that the material defining the pleats can be straightened as tension is applied across the body 102 or as any other force (e.g., a force applied at an angle non-parallel to the surface of the body 102) causes the body 102 to release the stored length. Alternatively, the material defining the pleats can be straightened in response to the absence of tension being applied across the body or of any other force applied to the body 102, or to the opening or hole 104. In some examples, the straightening of the pleats may cause the body 102 to transition from the first configuration of FIG. 1A to the second configuration of FIG. 1B.

In some embodiments, the body 102 includes from about 30% to about 40% stored length, from about 40% to about 50% stored length, from about 50% to about 60% stored length, from about 60% to about 70% stored length, from about 70% to about 80% stored length, from about 80% to about 90% stored length, from about 90% to about 100% stored length, from about 100% to about 110% stored length, from about 110% to about 120% stored length, and from about 120% to about 130% stored length, or any value or range between the foregoing values, for example.

In some embodiments, the body 102 is at least uniaxially or biaxially expandable (as shown in FIG. 1A, for example) at room temperature. Multi- (e.g., bi-) radial expansibility may be facilitated by incorporating pleating that stores length in multiple (e.g., at least two) directions. For example, macro-pleats can be oriented in various direction which allows stored length to be incorporated, and then released in various directions. Biaxial expansion may be desired in various instances, such as when material of the body 102 is anchored to tissue around its periphery and the material is being expanded by application of force(s) along one of the faces of the material. By providing biaxial expansion, stresses at the anchor points may be reduced during expansion as compared to uniaxial expansion.

In some embodiments, the body 102 may define regions of greater and lesser stored length. The relative amounts of stored length in these regions can be tuned by modifying pleating arrangements, materials, and other characteristics. For example, the body 102 may include at least two regions (e.g., a first region and a second region) having differing stored length properties. The first region may include less stored length per cm² than the second region. This allows for the various regions to preferentially act as less deformable support regions with less expansion and other regions to expand to accommodate shape change and stresses during use. It is understood that the amount of stored length in each region and the number of regions can be tuned to specific applications.

In some examples, the pleats (e.g., macro or micro-pleats) may store length, which in turn facilitates expansion of the material forming the body 102. The pleats may include different sizes and spacing. In some examples, the body 102 may include micro-pleats that are positioned from 0.05 mm to 0.10 mm, from 0.10 mm to 0.15 mm, or any other suitable range of value therebetween, from each other. The pleats are defined between folds represented by the spaces. The folds define portions of the body 102 that are turning inward (partially or fully) within a Z-axis of the body 102. Portions of the body 102 positioned within the folds may be configured to unfold or be pulled out such that those portions define an outer surface of the body 102. In some examples, the pleats may be formed on both surfaces (for example, upper and lower, or inner and outer, surfaces) of the body 102, as well as or alternatively or in addition to, within the substrate of the body 102. In some embodiments, having pleats on both surfaces may result in the body 102 being positioned to include a substantially serpentine or “S” shape. The serpentine shape is largely defined by the filaments or fibers forming the body 102. The serpentine shape may not necessarily be defined by a single filament or length of a filament, but may be taken as an average or bulk property of the filament(s) forming a length of the body 102.

As discussed the pleats and regions of the material of the body 102 may be tuned to have different amounts of stored length. Thus, the examples provided are just that, examples. Other ranges for distances between pleats can be from 0 to 10 μm, from 10 to 20 μm, from 20 to 30 μm, from 30 to 40 μm, from 40 to 50 μm, from 50 to 60 μm, from 60 to 70 μm, from 70 to 80 μm, from 80 to 90 μm, from 90 to 100 μm, from 100 to 200 μm, from 200 to 300 μm, from 300 to 400 μm, from 400 to 500 μm, from 500 to 600 μm, from 600 to 700 μm, from 700 to 800 μm, from 800 to 900 μm, from 900 to 1000 μm, from 1.0 to 1.5 mm, from 1.5 to 2.0 mm, from 2.0 to 3.0 mm, from 3.0 to 4.0 mm, from 4.0 to 5.0 mm, from 5.0 to 6.0 mm, or any other suitable value or range therebetween, or any suitable combination of ranges thereof.

In some examples, the pleats and folds of the body 102 may intersect each other. The intersections of the pleats may be a result of the shape and orientation of the pleats. For example, the pleats, in some embodiments, may not extend in a straight course along the surface of the body 102. Instead, the pleats may take a non-linear or non-straight path across the surface of the body 102. In some instances a pleat may not extend fully across a surface of the body 102, but instead extends across only a portion of the body 102. The pleats may intersect each other such that they combine into a single pleat or such that one pleat terminates at a position of a second pleat. The pleats can include any number of shapes and orientations including “S” shaped, lobed, irregular, linear, and so forth. In some embodiments, the shapes are irregular but include substantially similar sizes. In some instances, the surface of the body 102 may have the appearance of the folds or wrinkles of the surface of a human brain (e.g., similar shapes to sulci and gyri) or the surface of a walnut. In these embodiments, the pleats and folds may be positioned on a body 102 that defines a two-dimensional structure (e.g., a flat sheet) or a three-dimensional structure. Stated otherwise, the pleats and folds define a network or web of pleats and folds. The intersections and shapes of the pleats and folds as described facilitate stored length in the material in the X-axis (defined generally by the surface of the body 102) and the Y-axis (defined generally by the surface of the body 102), and combinations thereof.

Because the stored length is facilitated by the pleats and folds, it is generally understood that during expansion or release of the pleats and folds as previously discussed, the thickness of the body 102 decreases as the stored length is released. However, during release of the stored length, the thickness or structure of the body 102 is generally consistent. For example, the body 102 may include a microstructure defined by the filaments, where the microstructure is substantially uncollapsed during expansion. This allows the body 102 to retain many of its properties and functionalities such as porosity, pore size, plushness, cellular ingrowth, and so forth. The microstructure of the body 102 is described in more detail hereafter.

In some embodiments, in order to define the pleats and folds of the body 102, the filaments defining the body 102 may include melt-formed continuous filaments intermingled to form a porous web, wherein the melt-formed continuous filaments are self-cohered to each other at multiple contact points. Those filaments may be laid to form both the microstructure of the body 102 as well as the pleats and folds (e.g., via three-dimensional printing) or the microstructure may be formed and then the body 102 may be processed according to methods discussed herein to define the pleats and folds. In some examples, the melt-formed continuous filaments include at least one semi-crystalline polymeric component covalently bonded to or blended with at least one amorphous polymeric component. The melt-formed continuous filaments may possess partial to full polymeric component phase immiscibility when in a crystalline state. In some embodiments, once the pleats and folds are defined by the body 102, the body 102 can be processed or treated to instigate the crystalline state of at least some of the filaments. Otherwise stated, the orientation of the filaments can be set in the crystalline state to substantially retain the shape of the pleats and folds.

In some embodiments, the body 102 may be provided with various porosities. For example, in some embodiments, the body 102 may have a percent porosity greater than 70%, greater than 80%, greater than 90%, or any other suitable range therebetween. The porosity of the body 102 is defined within the three-dimensional microstructure. Because the body 102 includes a thickness that is greater than a single filament or even multiple stacked filaments, the pores are defined through a thickness of the body 102. In some embodiments, the body 102 may be provided with various thicknesses. For example, in some embodiments, the body 102 may be at least 0.10 mm thick, at least 0.20 mm thick, at least 0.30 mm thick, at least 0.40 mm thick, at least 0.50 mm thick, at least 0.60 mm thick, at least 0.70 mm thick, at least 0.80 mm thick, at least 0.90 mm thick, at least 1.00 mm thick, at least 2.00 mm thick, or at least 3.00 mm thick. The three-dimensional microstructure of the body 102 may include a network of pores throughout, including throughout the thickness of the body 102. Because of the three-dimensional character of the body 102 and the porous structure defined therein, the body 102 may provide various functionalities. For example, when used in surgical applications, the three-dimensional porous structure may facilitate tissue ingrowth and incorporation, which can provide for faster healing, increased adhesion, and so forth.

In addition to the body 102 including a three-dimensional structure through the thickness (e.g., Z-axis), in some embodiments the body 102 can be provided with a bulk, or overall three-dimensional shape or structure. For example, the body 102 may define a three-dimensional structure useful for the application in which it is to be used. The three-dimensional structure may include at least one of: a tubular construct, a sphere, a hemisphere, a partial sphere, a spheroid, a hemispheroid, a partial spheroid, an ellipsoid, a hemi-ellipsoid, a partial ellipsoid, a cone, and a partial dome. In other embodiments, the body 102 may be provided as a sheet having a substantially planar structure.

In some examples, the stored length of the body 102 is recoverable stored length. When the hole 104 of the body 102 is expanded and released, the hole 104 is capable of recovering at least some of the stored length. For example, the pleats and folds are at least partially recovered after expansion. In some embodiments, not all of the original stored length is recovered after the first expansion. In some embodiments, the body 102 demonstrates substantially the same recovery of stored length following expansion and release cycle over a plurality of subsequent cycles. In other words, the article retains its recovery properties, and the recovery property remains substantially the same, over a plurality of repetitions or cycles. In some embodiments, the stored length of the material is facilitated by the orientation of the filaments and specifically the orientation of the filaments being at least partially locked into the crystalline state of the material.

Bioabsorbable filaments may be implemented in connection with the processes described therein and may result in substrates implemented in further processing as described herein.

The substrates may generally be processed to provide stored length and micro-pleats as described herein. The processing methods may include positioning the body 102 between two sheets of material, and setting the non-woven member in the shape (e.g., inducing the crystalline state). In some embodiments, the method may further include positioning the body 102 with a stretched elastomeric material during the processing of the body 102 (e.g., on one or both sizes of the body to be treated) and then releasing the elastomeric material to a non-stretched configuration, wherein the elastomeric material facilitates the formation or definition of the pleats and folds in the body 102. Regions of differing stored length may be implemented by stretching the elastomeric material more or less across portions of the elastomeric material.

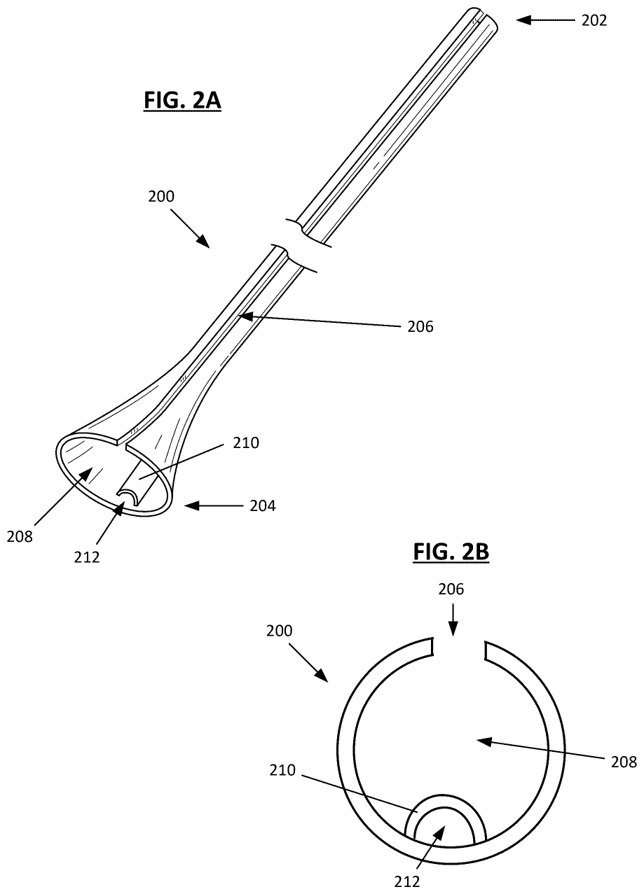

FIGS. 2A and 2B show an embodiment of a pusher sleeve 200 that is capable of being engaged with and slidingly received on an introducer as further discussed herein. An introducer is an elongated tubular member that allows access to vasculature as is known in the art. The pusher sleeve 200 has a first end 202 and a second end 204, as well as a slit 206 and a main channel 208 that extends longitudinally from the first end 202 to the second end 204. In some examples, the slit 206 is omitted. The main channel 208 is operable to accept the introducer therein. The slit 206 may be open or openable (that is, transitionable between open and closed configurations), in other words, defining variable widths. In FIGS. 2A and 2B, the slit 206 is shown in an open configuration. The second end 204 is the portion of the pusher sleeve 200 that comes into contact with the body 102 of the closure device 100. The slit 206 may be formed by initially forming the pusher sleeve 200 as a tubular construct using any suitable polymeric material, and subsequently cutting or slicing through the wall of the tubular construct along a longitudinal direction of the tubular construct. In some examples, the slit 206 may remain closed (so as to form a closed main channel 208) unless a force is applied to open the slit 206 (which also causes the main channel 208 to open). In some examples, the second end 204 may be wider than the first end 202 (for example, forming a substantially tapered, bell-shaped, or funnel-shaped configuration) such that the second end 204 may better engage with or support the body 102 while pushing the body 102 of the closure device 100 to the vasculature. The slit 206 generally facilitates expansion of the main channel 208 of the pusher sleeve 200. Additionally or alternatively, the pusher sleeve 200 may include elastomeric features to facilitate expansion of the main channel 208 of the pusher sleeve 200. For example, the pusher sleeve 200 may not include a slit, but instead be capable of being elastically (or, alternatively, inelastically) expanded when received over an introducer to increase the dimensions (e.g., diameter) of the main channel 208.

In some examples, the pusher sleeve 200 includes an inner conduit 210 that defines an inner channel 212 which extends longitudinally from the first end 202 to the second end 204 independently of the main channel 208. That is, the channels 208 and 212 are independent of and not in fluid communication with each other; the channels are separated from each other such that different objects or fluids may pass through one of the channels without affecting the other channel. The inner channel 212 occupies a smaller portion of an entire volume within the pusher sleeve 200 than does the main channel 208. The inner channel 212 may be configured to facilitate the transfer or transport of fluid such as adhesive polymer or chemical to be delivered to the body 102 of the closure device 100, whereas the main channel 208 may be configured to allow the introducer to pass through.

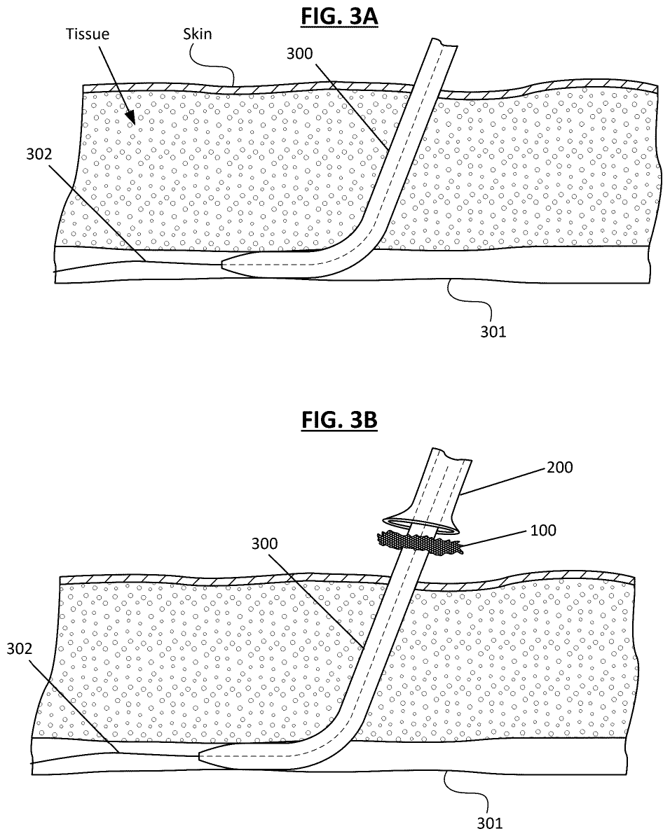

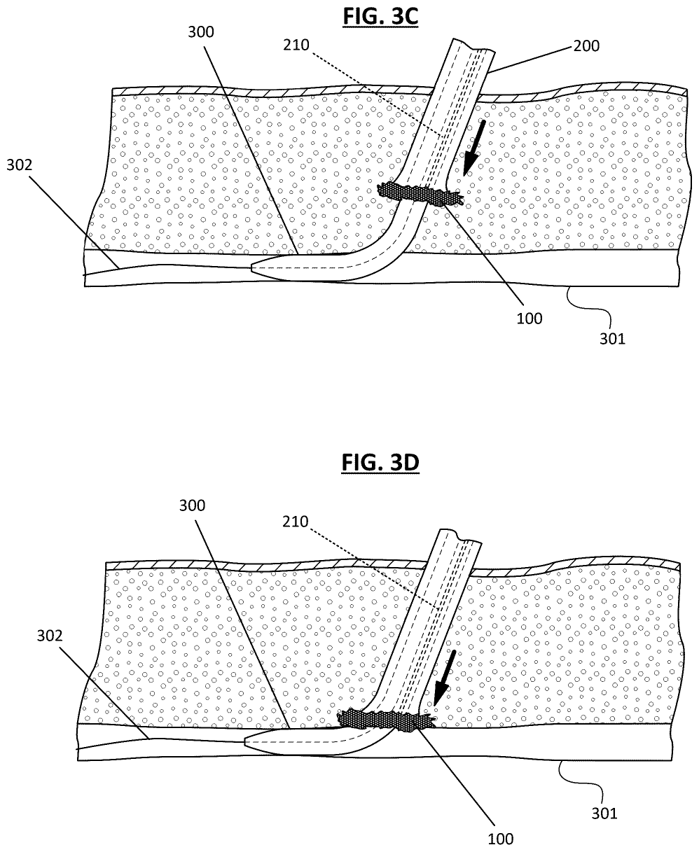

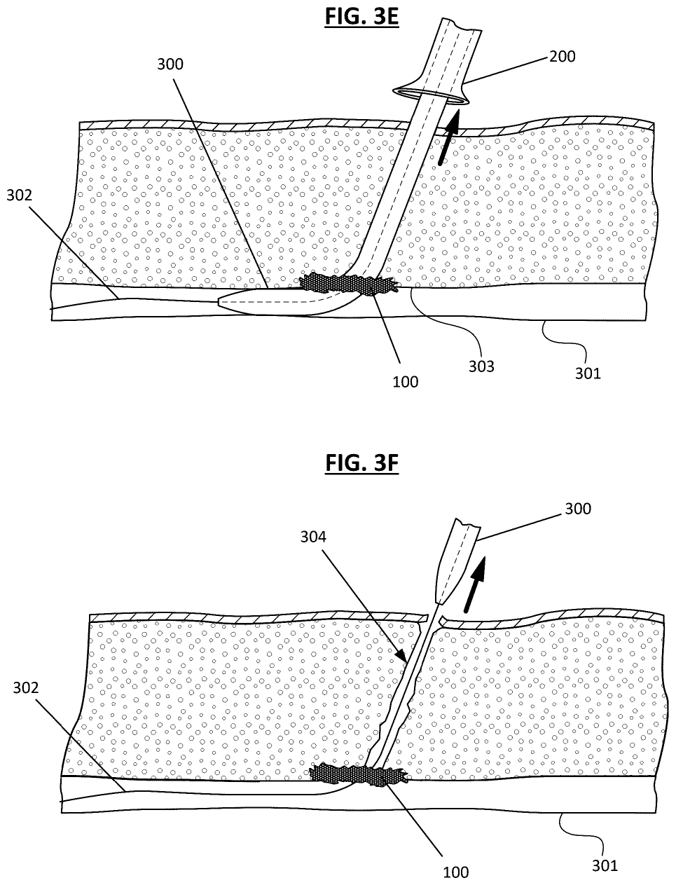

FIGS. 3A through 3F illustrate a procedure which may be implemented using the closure device 100 and the pusher sleeve 200 so as to perform a procedure in a vasculature and subsequently sealing the entry point for the surgical apparatus.

In FIG. 3A, a guidewire 302 is directed to pass into the vessel or artery of the body. The guidewire 302 provides access for additional medical devices to be transported along the guidewire and into the vessel or artery, and as such, the guidewire 302 may be used to direct any suitable medical devices, including but not limited to stent-grafts, toward the target location with the vasculature 301. An introducer 300 may also be directed into the vessel or artery along the guidewire 302, by passing distally along the guidewire 302 such that the guidewire 302 is disposed inside an internal channel of the introducer 300. Specifically, an introducer 300 is inserted into the body through the skin and the tissue underneath along the guidewire 302, as shown, until a distal tip of the introducer 300 reaches a vasculature 301 such as a vessel, which may be a blood vessel such as a vein or an artery, thus forming an access path to the vasculature 301. The introducer 300 may be operable to penetrate through a tissue region adjacent to the vasculature 301 to form a tissue tract 304. The introducer 300 may be directed in either direction of the fluid flow within the vessel, such as blood flow. For example, the introducer 500 may be directed or angled such that the distal tip of the introducer 500 is directed in an opposite direction from the blood flow (retrograde) or in the same direction as blood flow (antegrade). The introducer 300 allows a guidewire 302 (as well as other implantable medical devices which are not shown) to pass through an internal channel of the introducer 300 and into the vessel (e.g., vein or artery). The guidewire 302 may be used to direct any suitable medical devices, including but not limited to stent-grafts, toward the target location with the vasculature 301.

In FIG. 3B, the pusher sleeve 200 is disposed on the introducer 300, and the introducer 300 is passed through the hole 104 of the closure device 100, such that the closure device 100 and the pusher sleeve 200 are both disposed on the introducer 300, and the closure device 100 is disposed distally (that is, further from the physician or clinician who is performing the surgical operation) than the pusher sleeve 200, with the second end 204 of the pusher sleeve 200 disposed so as to apply a generally axial force upon the closure device 100 by the pusher sleeve 200 in urging engagement therewith.

In FIG. 3C, the pusher sleeve 200 is operable to push the closure device 100 distally along the body of the introducer 300 into the body of the patient and toward the vasculature therein, as shown by the arrow.

In FIG. 3D, the closure device 100 is advanced to reach the intersection of the vasculature 301 (vein or artery) and the access path. In some examples, the pusher sleeve 200 may include the inner conduit 210 through which adhesive fluid is passed such that the adhesive fluid is delivered to the closure device 100 at the intersection while the closure device 100 is maintained at the location. As such, beneficially, the adhesive material may be delivered without having to insert another catheter or conduit to do so, and the pusher sleeve 200 can operate to both hold the closure device 100 in place and deliver the adhesive while being disposed around the introducer 300.

Additionally or alternatively, the adhesive 108 that is disposed on a surface of the body 102 may be a biocompatible adhesive that is compatible with blood or saline solution. In some examples, the adhesive 108 may be provided along the way toward the stage as shown in FIG. 3D, when the closure device 100 is in a blood-exposed environment. The adhesive 108 may be applied between the body 102 of the closure device 100 and the surrounding tissue wall 303 of the vasculature 301 (vein or artery) such that the adhesive 108 causes the body 102 to be attached to the tissue wall 303 surrounding the tissue tract 304.

In FIG. 3E, when the adhesive is properly applied so that the closure device 100 is adhered or attached to the desired location, such as the aforementioned intersection of the vasculature 301 (vein or artery) and the access path, the pusher sleeve 200 is then retracted along the introducer 300 in the direction shown by the arrow, while the introducer 300 is still maintained in its initial position at FIG. 3A. In some examples, the slit 206 is opened to allow for the introducer 300 to pass through, such as when the pusher sleeve 200 is being removed from about the introducer 300. This may be beneficial when it is difficult or unreasonable to longitudinally retract the pusher sleeve 200 for the entire length of the introducer 300. Instead, by opening the slit 206, the pusher sleeve 200 can be removed from the introducer 300 more easily by first uncoupling from the introducer 300 such that the pusher sleeve 200 can be retrieved independently and/or separately from the introducer 300. The benefit of decoupling the pusher sleeve 200 from the introducer 300 may include allowing the introducer 300 to remain undisturbed while the pusher sleeve 200 is being removed.

In FIG. 3F, after the surgical procedure that required vascular access is completed, the introducer 300 is retracted from the access path (now shown as tissue tract 304) in the direction shown by the arrow, while the guidewire 302 remains in the vasculature, and the closure device 100 operates to close the entryway to the tissue tract 304 by the closing of the hole 104 of the closure device 100, effectively self-sealing the hole 104 by transitioning between the first configuration and the second configuration in response to the absence of the force applied by the introducer 300 that caused the hole 104 to expand. The closure device 100 may be reduced in width due to the pressure applied to the body 102 of the closure device 100 from the surrounding tissue wall 303.

In some examples, the guidewire 302 may then be retracted from the vasculature and from the tissue tract 304, leaving only the closure device 100 (and any medical device, not shown, that is implanted as part of the surgical procedure) inside the body of the patient. In some examples, alternatively, the guidewire 302 may be left in place, should the physician or clinician need to regain access to the vasculature at a later time.

In some examples, the self-sealing of the closure device 100 may result in at least 80%, at least 85%, at least 90%, or at least 95% reduction in the amount of fluid that is allowed to flow through the hole 104 or through the body 102 while in the second (or substantially closed) configuration. In some embodiments, a predetermined leakage of fluid through the hole 104 is effectuated.

In some examples, the body 102 may be formed so as to facilitate tissue ingrowth into the body 102 when implanted as shown in FIG. 3F, such that the tissue ingrowth over time further reduces the amount of fluid that is allowed to flow through the body 102 between the vasculature 301 (vein or artery) and the tissue tract 304.

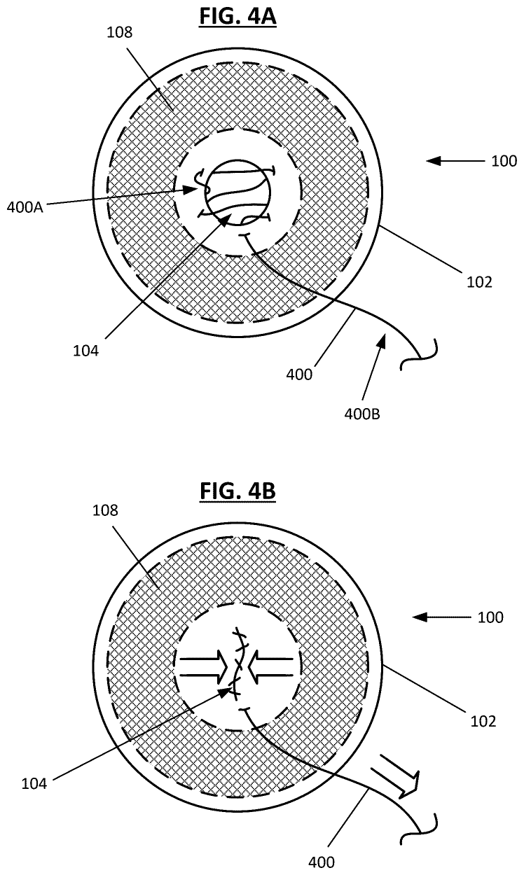

FIGS. 4A and 4B show another example of the closure device 100 according to some embodiments disclosed herein, in which actuating an extension 400 may cause the hole 104 to be actuated between the first (open) configuration and the second (substantially closed) configuration. In addition to the body 102 having the hole 104, the closure device 100 also includes an extension 400 that extends from the body 102 and weaves through the body 102 and across the hole 104 such that the extension 400 extends in a cross-cross pattern to and from the opposing sides of the hole 104 in the body 102, as shown in FIG. 4A, when the closure device 100 is in the first (open) configuration.

In FIG. 4B, the closure device 100 is in the second (substantially closed) configuration when the extension 400 is actuated (e.g., pulled proximally as shown by the arrow along the extension 400), and the actuation (e.g., pulling, tensioning, or tightening) of the extension 400 causes the opposing ends of the hole 104 to approach each other as shown by the two arrows facing each other, resulting in the hole 104 to be closed or substantially closed, resembling a sewn stitch that is formed on the body 102. As such, in the example shown, the closure device 100 is not self-sealing but is capable of sealing in response to the tightening of the extension 400.

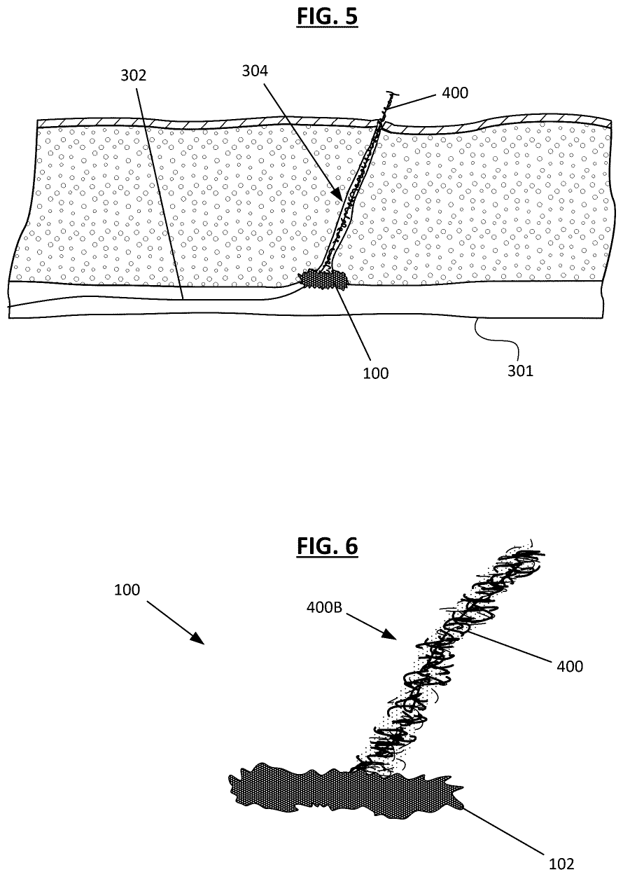

FIG. 5 shows an example of how the extension 400 may be disposed with respect to the vasculature 301 (vein or artery) and the tissue tract 304. Procedurally, FIG. 5 takes place after the introducer 300 is fully retracted as per FIG. 3E. However, instead of having only the guidewire 302 extending through the tissue tract 304, this example also includes the extension 400 extending through the tissue tract 304, such that the tissue tract 304 is occupied by both the guidewire 302 and the extension 400 attached to the body 102 of the closure device 100. In this example, the extension 400 has been pulled or tightened such that the hole 104 of the body 102 is substantially closed in the second (closed) configuration, as shown in FIG. 4B. Furthermore, the extension 400 may be configured to fill at least a portion of the tissue tract 304, such that tissue ingrowth may be facilitated into the extension 400, which beneficially further fills the tissue tract 304 to further reduce the risk of fluid leakage from the vasculature 301 (vein or artery).

FIG. 6 shows an example of the closure device 100 in which the extension 400 is configured to increase the amount or efficiency of tissue ingrowth within the structure of the extension 400. For example, the extension 400 may be formed as a fluffy or “fuzzy” construct, such as one resembling a collection (or network) of interconnected fibrils forming a composite (or network) of various separate fibrils or filaments, which may be in any suitable ways, such as in a braided, latticed, or randomly intertwined configuration, among others. Due to the “fuzzy” property of the extension 400, the extension 400 may have numerous spaces or pockets into which tissue cells may enter to facilitate tissue ingrowth. In some examples, the extension 400 may be formed of a continuous filament or composite of filaments that are made of a single material.

In some examples, the extension 400 may be formed by attaching together (such as via tying or knotting together or via the use of an adhesive) two or more separate components that are made of different materials or have different physical properties. In some examples, a first portion 400A of the extension 400, which may be the portion that weaves through the body 102 and across the hole 104 as shown in FIG. 4A, and a second portion 400B of the extension 400, which may be the portion that extends outwardly from the body 102, may be made of two different materials or a single material with two different physical properties. For example, the first portion 400A may be formed using a material that is comprised of micro-pleats, and the second portion 400B may be formed using a material that is comprised of macro-pleats. Generally, inclusion of macro-pleats facilitates more tissue ingrowth than micro-pleats, when using the same material to form both types of pleats. In some examples, the body 102 may be formed using the material that is comprised of micro-pleats, in which case the body 102 may facilitate less tissue ingrowth as compared to the second portion 400B.

The differences in the pleat sizes contributes to the amount of space that is available for tissue ingrowth. For example, the material with macro-pleats is more susceptible to tissue ingrowth, and as such, the material with macro-pleats is more suitable to be located within the tissue tract 304 than the material with micro-pleats. Also, the material with micro-pleats may be more suitable to sew the hole 104 closed, because the material with macro-pleats may be too thick or coarse to be effectively used for stitching.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An implantable closure device comprising:
a body comprising a bioabsorbable material and defining generally a disk-shape having a hole wherein the body is distensible so as to define the hole having an open position when the hole is presented with a force and a closed position when the force is absent.

2. The implantable closure device of claim 1, further comprising an adhesive applied to the body.

3. The implantable closure device of claim 2, wherein the adhesive is a biocompatible adhesive.

4. The implantable closure device of claim 1, further comprising an extension weaved through the body and extending from the body, wherein actuating the extension causes the hole to be actuated between the open position and the closed position.

5. The implantable closure device of claim 4, wherein the extension comprises a network of interconnected fibrils.

6. The implantable closure device of claim 4, wherein the extension comprises: a first portion with micro-pleats that is weaved through the body, and a second portion with macro-pleats that is extending from the body.

7. The implantable closure device of claim 1, wherein the body is radially distensible.

8. The implantable closure device of claim 1, wherein the bioabsorbable material has a property of stored length operable to effectuate recovery of the hole from the open position to the closed position.

9. The implantable closure device of claim 8, wherein the stored length is facilitated by defining the bioabsorbable material with micro-pleats that allow for elongation and contraction.

10. A surgical apparatus for implanting and closing a vasculature in a body of a patient, the apparatus comprising:
an implantable closure device comprising a body having a bioabsorbable material and defining generally a disk-shape having a hole wherein the body is distensible so as to define the hole having an open position when the hole is presented with a force and a closed position when the force is absent;
an introducer operable to penetrate through a tissue region adjacent to the vasculature to form a tissue tract; and
a pusher sleeve having a main channel operable to accept the introducer therein, the pusher sleeve having a first end and a second end, wherein the second end is operable to engage the implantable closure device during implanting the implantable closure device adjacent to the vasculature to be closed.

11. The surgical apparatus of claim 10, wherein the pusher sleeve includes an openable slit extending longitudinally from the first end to the second end of the pusher sleeve operable to allow the passing of the introducer therethrough to facilitate the coupling of the pusher sleeve onto the introducer.

12. The surgical apparatus of claim 10, wherein the pusher sleeve includes an inner conduit that defines an inner channel extending longitudinally from the first end to the second end independent of and not in fluid communication with the main channel.

13. The surgical apparatus of claim 12, wherein the inner channel is operable to facilitate the transfer of an adhesive material to the implantable closure device.

14. The surgical apparatus of claim 10, wherein the implantable closure device includes an extension weaved through the body and extending from the body, actuating the extension causes the hole to be actuated between the open position and the closed position, the extension comprises a first portion with micro-pleats that is weaved through the body and a second portion with macro-pleats that is extending from the body, and the second portion of the extension extends through the tissue tract to facilitate cell ingrowth.

15. The surgical apparatus of claim 10, further comprising a guidewire operable to extend through the introducer.

16. A method of implanting and closing a vasculature in a body of a patient, the method comprising:
extending an introducer through a hole in an implantable closure device having a body comprising a bioabsorbable material, the hole operable to be distensible in one or more directions by the urging engagement of the introducer therein having a larger diameter into the hole having a smaller diameter;
extending the introducer through a tissue tract formed in a tissue region adjacent to the vasculature;
advancing the implantable closure device toward the vasculature via a pusher sleeve to adjacent to an in contact with the vasculature, the pusher sleeve having a main channel operable to be received onto the introducer therein;
retracting the pusher sleeve from the tissue tract; and
retracting the introducer from the tissue tract and the implantable closure device causing the hole of the implantable closure device to self-transition from an open position when the introducer is located within the hole to a closed position when the introducer is withdrawn from the hole in an absence of a force being applied to open the hole.

17. The method of claim 16, wherein the retraction of the introducer from the tissue tract causes a second portion of an extension to extend from the implantable closure device through the tissue tract to facilitate cell ingrowth, wherein a first portion of the extension is weaved through the body and across the hole such that the first portion of the extension extends in a cross-cross pattern to and from opposing sides of the hole of the implantable closure device.

18. The method of claim 17, further comprising:
tensioning the extension, wherein the tensioning the extension is operable to cause the first portion of the extension to substantially close the hole of the implantable closure device.

19. The method of claim 16, wherein the pusher sleeve includes an openable slit extending longitudinally along the pusher sleeve, the method further comprising:
opening the openable slit of the pusher sleeve to retrieve the pusher sleeve from the tissue tract independently of the introducer.

20. The method of claim 16, further comprising: prior to the retraction of the pusher sleeve, delivering an adhesive to the implantable closure device to couple the implantable closure device to the vasculature and/or adjacent tissue.
